(19) United States
(12) Patent Application Publication
(10) Pub. No.: US 2023/0404740 A1
(43) Pub. Date: Dec. 21, 2023

(54) INTRAOCULAR LENS

(71) Applicant: DONGGUAN AIER OPTHALMOLOGY HOSPITAL CO., LTD., Dongguan (CN)

(72) Inventor: Fei LIU, Dongguan (CN)

(73) Assignee: DONGGUAN AIER OPTHALMOLOGY HOSPITAL CO., LTD., Dongguan (CN)

(21) Appl. No.: 18/099,923

(22) Filed: Jan. 21, 2023

(30) Foreign Application Priority Data
Jun. 21, 2022 (CN) 202210706418.9
Jun. 21, 2022 (CN) 202221566011.2

(51) Int. Cl. A61F 2/16

(52) U.S. Cl. CPC A61F 2/1618

(57) ABSTRACT

Disclosed is an intraocular lens, including an optical portion. A first surface of the optical portion includes a first central area and a first annular area, which have different focal powers; a second surface of the optical portion includes a second central area and a second annular area, which have different focal powers, and the first central area and the second central area are not completely projected onto each other. Based on the ability of the areas on the first surface and the areas on the second surface of the optical portion to refract the light, the light enters into the optical portion from the first surface, and is refracted from the second surface of the optical portion, and converged to at least three different positions on the optical axis of the optical portion.

This disclosure claims priorities to the following two Chinese patent applications, both of which are incorporated herein by reference in their entireties, 1) Chinese Patent Application No. 202210706418.9, titled “INTRAOCULAR LENS”, filed with the China National Intellectual Property Administration on Jun. 21, 2022, and 2) Chinese Patent Application No. 202221566011.2, titled “INTRAOCULAR LENS”, filed with the China National Intellectual Property Administration on Jun. 21, 2022.

### FIELD

The present disclosure relates to the technical field of ophthalmic products, and in particular to an intraocular lens.

### BACKGROUND

The crystalline lens of the normal human eye is transparent, which is an important constituent part forming the total focal power of the eye and is biconvex. A curvature radius of an anterior surface is substantially 10 mm, a curvature radius of a posterior surface is substantially 6 mm, and the focal power of the crystalline lens of the human eye is variable, such as a zoom lens of a camera. The crystalline lens of the human eye is elastic. The crystalline lens becomes thinner when looking at an object at a far distance, and the focal power accordingly becomes less. The crystalline lens becomes thicker when looking at objects at a proximal distance, and the focal power accordingly becomes greater, so that the light from the objects at different distance can be focused and imaged on the retina. The light transmittance is deteriorated after the crystalline lens becomes turbid, that is, cataract occurs. The current treatment method is to remove the cataract and implant an intraocular lens with a certain focal power to replace the original crystalline lens.

The early implanted intraocular lens is monofocal and cannot zoom like the crystalline lens, which leads to clear distant vision and poor proximal and medium vision after cataract surgery. For example, a pair of presbyopia glasses needs to be worn to see objects at the proximal distance. Subsequently, a multifocal intraocular lens is developed, which includes a refractive multifocal intraocular lens and a diffractive multifocal intraocular lens. However, the conventional refractive multifocal intraocular lens has uneven refractive rings on its surface, which is easy to cause glare and halo, requires high precision on the central position of the intraocular lens after implantation, and a slight deviation is easy to cause vertigo. The diffractive multifocal intraocular lens based on the principle of diffractive optics has uneven diffractive rings on its surface, which belongs to Fresnel lens, has a poor imaging quality is poor, and may produce glare, halo and large loss of light energy.

### SUMMARY

In view of this, an intraocular lens is provided according to the present disclosure, which includes at least three focuses, and thus is not easy to generate glare and halo and has small loss of light energy compared with the prior art.

In order to achieve the above object, the following technical solution is provided according to the present disclosure:

an intraocular lens includes an optical portion, a first surface of the optical portion includes a first central area and at least one first annular area, and the first central area and the at least one first annular area have different focal powers;

a second surface of the optical portion includes a second central area and at least one second annular area, the second central area and the at least one second annular area have different focal powers, the first central area and the second central area are not completely projected onto each other, so that light enters into the optical portion from the first surface, and is refracted from the second surface, and converged to at least three different positions on an optical axis of the optical portion.

Preferably, the light from a first-distance object enters into the optical portion from the first central area, and is refracted from the second central area, and converged to a first position on the optical axis of the optical portion;

if the second central area is not completely projected onto the first central area, the light from a second-distance object enters into the optical portion from the at least one first annular area, and is refracted from the at least one second central area, and converged to a second position on the optical axis of the optical portion;

if the first central area is not completely projected onto the second central area, the light from the second-distance object enters into the optical portion from the first central area, and is refracted from the second annular area, and converged to the second position on the optical axis of the optical portion;

a distance from the first-distance object to the center of the intraocular lens is different from a distance from the second-distance object to the center of the intraocular lens.

Preferably, the distance from the first-distance object to the center of the intraocular lens is greater than the distance from the second-distance object to the center of the intraocular lens.

Preferably, the light from a third-distance object enters into the optical portion from the at least one first annular area, refracted from the at least one second annular area, converged to a third position on the optical axis of the optical portion, and a distance from the third-distance object to the center of the intraocular lens is different from both the distance from the second-distance object to the center of the intraocular lens and the distance from the first-distance object to the center of the intraocular lens.

Preferably, the distance from the first-distance object to the center of the intraocular lens is greater than the distance from the second-distance object to the center of the intraocular lens, and the distance from the second-distance object to the center of the intraocular lens is greater than the distance from the third-distance object to the center of the intraocular lens.

Preferably, a distance from the second position to the center of the intraocular lens is less than a distance from the first position to the center of the intraocular lens, and a distance from the third position to the center of the intraocular lens is less than a distance from the second position to the center of the intraocular lens.

Preferably, the second central area is not completely projected onto the first central area, the first surface of the optical portion includes at least two first annular areas, the first annular area adjacent to the first central area is not completely projected onto the second central area, and each of the at least two first annular areas has different focal powers;

or, the first central area is not completely projected onto the second central area, the second surface of the optical portion includes at least two second annular areas, and the second annular area adjacent to the second central area is not completely projected onto the first central area, and each of the at least two second annular areas has different focal powers.

Preferably, the second central area is not completely projected onto the first central area, the first surface of the optical portion comprises a proximal first annular area and a far first annular area, and the proximal first annular area is not completely projected onto the second central area;

the light from a first-distance object enters into the optical portion from the first central area, and is refracted from the second central area, and converged to a first position on the optical axis of the optical portion;

the light from a second-distance object enters into the optical portion from the proximal first annular area, and is refracted from the second central area, and converged to a second position on the optical axis of the optical portion;

the light from a third-distance object enters into the optical portion from the proximal first annular area, and is refracted from the second annular area, and converged to a third position on the optical axis of the optical portion;

the light from the second-distance object enters into the optical portion from the far first annular area, and is refracted from the second annular area, and converged to the second position on the optical axis of the optical portion;

a distance from the first-distance object to the center of the intraocular lens, a distance from the second-distance object to the center of the intraocular lens and a distance from the third-distance object to the center of the intraocular lens are different.

Preferably, the first central area is not completely projected onto the second central area, the second surface of the optical portion includes a proximal second annular area and a far second annular area, and the proximal second annular area is not completely projected onto the first central area;

the light from a first-distance object enters into the optical portion from the first central area, and is refracted from the second central area, and converged to a first position on the optical axis of the optical portion;

the light from a second-distance object enters into the optical portion from the first central area, and is refracted from the proximal second annular area, and converged to a second position on the optical axis of the optical portion;

the light from a third-distance object enters into the optical portion from the first annular area, and is refracted from the proximal second annular area, and converged to a third position on the optical axis of the optical portion;

the light from the second-distance object enters into the optical portion from the first annular area, and is refracted from the far second annular area, and converged to the second position on the optical axis of the optical portion;

a distance from the first-distance object to the center of the intraocular lens, a distance from the second-distance object to the center of the intraocular lens and a distance from the third-distance object to the center of the intraocular lens are all different.

Preferably, a radial size of the first central area is less than a radial size of the second central area, so that the second central area is not completely projected onto the first central area;

or, a radial size of the first central area is greater than a radial size of the second central area, so that the first central area is not completely projected onto the second central area.

Preferably, a focal power of the at least one first annular area from an outer side to an inner side gradually increases, or, a focal power of the at least one second annular area from an outer side to an inner side gradually increases.

Preferably, an outer side edge of the first central area has a same height as an inner side edge of the at least one first annular area, or, an outer side edge of the at least one second central area has a same height as an inner side edge of the second annular area.

Preferably, the first central area is spherical or aspherical, the at least one first annular area is spherical or aspherical, the second central area is spherical or aspherical, and the at least one second annular area is spherical or aspherical.

It can be seen from the above technical solution that the intraocular lens provided according to the present disclosure includes the optical portion, a first surface of the optical portion includes a first central area and at least one first annular area, and the first central area and the at least one first annular area have different focal powers; the second surface of the optical portion includes a second central area and at least one second annular area, the second central area and the at least one second annular area have different focal powers, and the first central area and the second central area are not completely projected onto each other. Based on the ability of the areas on the first surface and the areas on the second surface of the optical portion to refract the light, the light enters into the optical portion from the first surface of the optical portion, and is refracted from the second surface of the optical portion, and converged to the at least three different positions on the optical axis of the optical portion. Therefore, the intraocular lens according to the present disclosure includes at least three focuses, which can provide different vision, and is not necessary to form multiple uneven annular structures on the surface of the intraocular lens compared with the prior art, so that the phenomenon of glare and halo is not easy to occur, and the loss of light energy is small.

### BRIEF DESCRIPTION OF THE DRAWINGS

For more clearly illustrating the technical solutions of embodiments of the present disclosure or in the conventional technology, drawings referred to for describing the embodiments or the conventional technology will be briefly described hereinafter. Apparently, the drawings in the following description are only several examples of the present disclosure, and for those skilled in the art, other drawings may be obtained based on these drawings without any creative efforts.

FIG. 1 is a front view of an intraocular lens provided according to an embodiment of the present disclosure;

FIG. 2 is an optical path diagram showing a first-distance object imaged by the intraocular lens shown in FIG. 1;

FIG. 3 is an optical path diagram showing a second-distance object imaged by the intraocular lens shown in FIG. 1;

FIG. 4 is an optical path diagram showing a third-distance object imaged by the intraocular lens shown in FIG. 1;

FIG. 5 is a front view of the intraocular lens provided according to another embodiment of the present disclosure; and

FIG. 6 is a side view of the intraocular lens shown in FIG. 5.

Reference numerals of the drawings in the specification are listed as follows:

| | |
|---|---|
| 10, | optical portion; |
| 11, | first surface; |
| 12, | second surface; |
| 100, | first central area; |
| 101, | first annular area; |
| 102, | second central area; |
| 103, | second annular area; |
| 104, | proximal first annular area; |
| 105, | far first annular area. |

### DETAILED DESCRIPTION OF THE EMBODIMENTS

For enabling those skilled in the art to better understand the technical solutions in the present disclosure, the technical solutions in the embodiments of the present disclosure will be described clearly and completely hereinafter in conjunction with the drawings in the embodiments of the present disclosure. It is apparent that the described embodiments are only a part of the embodiments of the present disclosure, rather than all embodiments. Based on the embodiments in the present disclosure, all of other embodiments, made by the person skilled in the art without any creative efforts, fall into the scope of the present disclosure.

An intraocular lens is provided according to an embodiment. The intraocular lens includes an optical portion, a first surface of the optical portion includes a first central area and at least one first annular area, and the first central area and the at least one first annular area have different focal powers; a second surface of the optical portion includes a second central area and at least one second annular area, the second central area and the at least one second annular area have different focal powers, the first central area and the second central area are not completely projected onto each other, so that light into the optical portion from the first surface is refracted from the second surface, and thus can be converged to at least three different positions on an optical axis of the optical portion.

The optical portion refers to a portion of the intraocular lens through which light needs to transmit. The focal power represents the ability of the optical system to refract the parallel light when the parallel light incidents on the optical system.

The light from the object enters into the optical portion from the first surface of the optical portion, and is refracted from the second surface of the optical portion for focusing imaging. Based on the ability of the areas on the first surface and the areas on the second surface of the optical portion to refract the light, the light enters into the optical portion from the first surface of the optical portion and then is refracted from the second surface of the optical portion, which can be converged to at least three different positions on the optical axis of the optical portion, that is, can perform focusing imaging at the at least three different positions on the optical axis of the optical portion. Therefore, the intraocular lens according to the embodiment includes at least three focuses, which can provide different vision, is not necessary to form multiple uneven annular structures on the surface of the intraocular lens compared with the prior art, so that the glare and halo are not easy to occur, and the loss of light energy is small.

In combination with the refraction of the first surface of the intraocular lens to the light and the refraction of the second surface to the light, focusing imaging is realized after the light transmits through the intraocular lens. In this embodiment, the focal powers of the first central area and the at least one first annular area of the first surface of the optical portion are not limited, and the focal powers of the second central area and the at least one second annular area of the second surface of the optical portion are not limited, which can be set according to the focus of the intraocular lens with different focal powers in practical application.

The first central area of the first surface and the second central area of the second surface are not completely projected onto each other, that is, there is a non-overlapping area between the first central area and the second central area. In this way, the light entering into the optical portion from the first central area is not all refracted from the second central area, and part of the light is refracted from the second annular area, so that this part of the light can be converged a different positions. Alternatively, not all the light refracted from the second central area enters into the optical portion from the first central area, and part of the light enters from the first annular area, so that the light can be converged to different positions. Therefore, the areas of the first surface of the intraocular lens according to the embodiment have different focal powers, the areas of the second surface have different focal powers, and the combination of the first surface and the second surface enables the intraocular lens to form multiple different focal powers, so that the intraocular lens has multiple focuses.

As an optional embodiment, the second central area is not completely projected onto the first central area, the light from a first-distance object enters into the optical portion from the first central area, is refracted from the second central area, and is converged to a first position on the optical axis of the optical portion; the light from a second-distance object enters into the optical portion from the first annular area, is refracted from the second central area, and is converged to a second position on the optical axis of the optical portion. A distance from the first-distance object to the center of the intraocular lens is different from a distance from the second-distance object to the center of the intraocular lens. The first-distance object refers to an object with a distance to the center of the intraocular lens within a first distance range, and the second-distance object refers to an object with a distance to the center of the intraocular lens within a second distance range.

In this embodiment, the magnitude relation between the distance from the first-distance object to the center of the intraocular lens and the distance from the second-distance object to the center of the intraocular lens is not limited. For example, a minimum distance of the first distance range may be greater than a maximum distance of the second distance range, that is, the distance from the first-distance object to the center of the intraocular lens is greater than the distance from the second-distance object to the center of the intraocular lens.

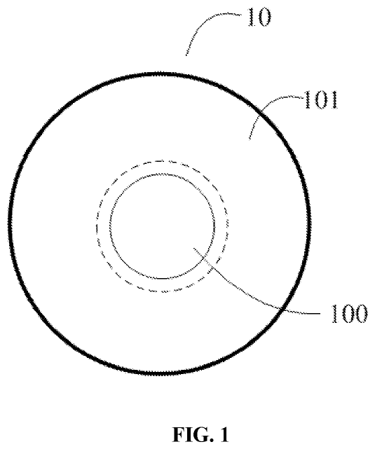

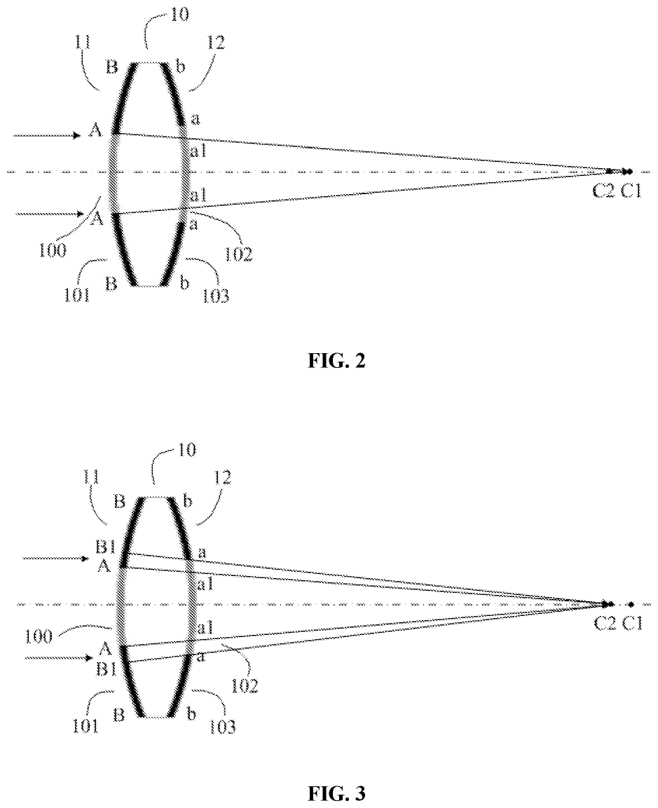

For example, referring to FIG. 1, FIG. 2 and FIG. 3, FIG. 1 is a front view of an intraocular lens provided according to an embodiment of the present disclosure; FIG. 2 is an optical path diagram showing a first-distance object imaged by the intraocular lens shown in FIG. 1; and FIG. 3 is an optical path diagram showing a second-distance object imaged by the intraocular lens shown in FIG. 1. As shown in FIG. 1, FIG. 2 and FIG. 3, a first surface 11 of the intraocular lens includes a first central area 100 and a first annular area 101, and a second surface 12 includes a second central area 102 and a second annular area 103. A radial size of the first central area 100 is less than a radial size of the second central area 102, so that the second central area 102 is not completely projected onto the first central area 100.

As shown in FIG. 2, the light from a first-distance object enters an optical portion 10 from the first central area 100, that is, from an A-A area, is refracted from the second central area 102, specifically refracted from an al-al area of the second central area 102, and is converged to a first position C1 on the optical axis of the optical portion 10. That is, the first-distance object is imaged at the first position C1 through the intraocular lens. As shown in FIG. 3, the light from a second-distance object enters into the optical portion 10 from the first annular area 101, specifically from an A-B1 area of the first annular area 101, is refracted from an al-a area of the second central area 102, and is converged to a second position C2 on the optical axis of the optical portion 10. That is, the second-distance object is imaged at the second position C2 through the intraocular lens. The intraocular lens in this embodiment has two focuses, which can image the first-distance object and the second-distance object.

In this embodiment, a distance from the second position C2 to the center of the intraocular lens is less than a distance from the first position C1 to the center of the intraocular lens. In practical application, the first position C1 can correspond to a position on the retina, and the second position C2 can correspond to a position in front of the retina.

Furthermore, based on the above embodiment, the light from a third-distance object enters into the optical portion from the first annular area, is refracted from the second annular area and converged to a third position on the optical axis of the optical portion. A distance from the third-distance object to the center of the intraocular lens is different from the distance from the second-distance object to the center of the intraocular lens and the distance from the first-distance object to the center of the intraocular lens. The third-distance object refers to an object with a distance to the center of the intraocular lens within a third distance range. In this embodiment, the magnitude relation among the distance from the third-distance object to the center of the intraocular lens, the distance from the first-distance object to the center of the intraocular lens and the distance from the second-distance object to the center of the intraocular lens is not limited. For example, the minimum distance of the first distance range may be greater than the maximum distance of the second distance range, a minimum distance of the second distance range may be greater than a maximum distance of the third distance range, that is, the distance from the first-distance object to the center of the intraocular lens is greater than the distance from the second-distance object to the center of the intraocular lens, and the distance from the second-distance object to the center of the intraocular lens is greater than the distance from the third-distance object to the center of the intraocular lens.

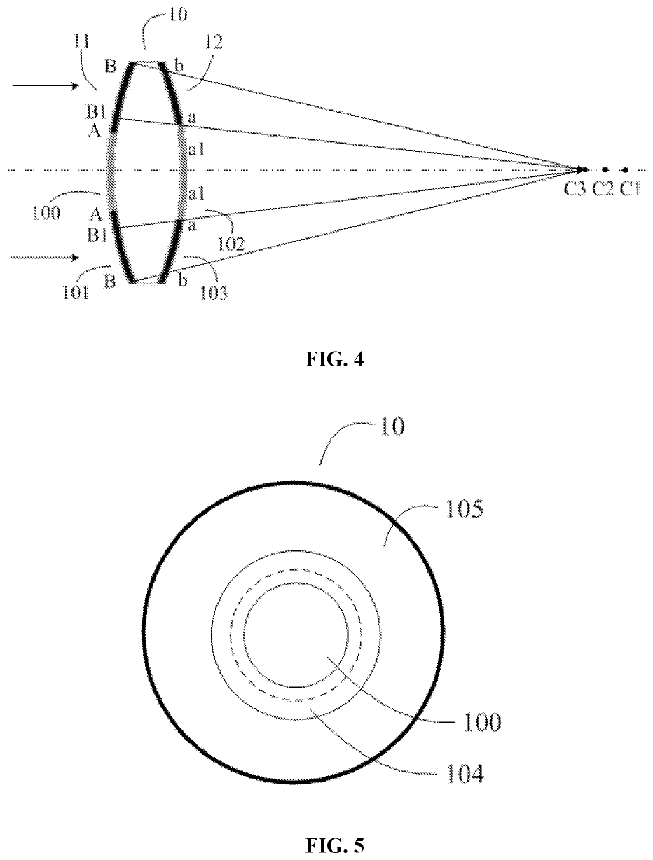

For example, referring to FIG. 4, FIG. 4 is an optical path diagram showing the third-distance object imaged by the intraocular lens shown in FIG. 1. As shown in FIG. 4, the light from the third-distance object enters into the optical portion 10 from the first annular area 101, specifically from a B1-B area of the first annular area 101, and is refracted from an a-b area of the second annular area 103 and converged to a third position C3 on the optical axis of the optical portion 10. That is, the third-distance object is imaged at the third position C3 through the intraocular lens. Therefore, the intraocular lens in this embodiment has three focuses, which can image the first-distance object, the second-distance object and the third-distance object, and can provide distant vision, medium vision and proximal vision.

In this embodiment, a distance from the third position C3 to the center of the intraocular lens is less than the distance from the second position C2 to the center of the intraocular lens. In practical application, the first position C1 can correspond to a position on the retina, and the third position C3 can correspond to a position in front of the retina.

Furthermore, in another optional embodiment, the second central area is not completely projected onto the first central area, the first surface of the optical portion includes at least two first annular areas, and the first annular area adjacent to the first central area is not completely projected onto the second central area, and these at least two first annular areas have different focal powers. The second central area is not completely projected onto the first central area, that is, at least part of the second central area is projected onto the first annular area adjacent to the first central area, and accordingly, the light into the optical portion from the first central area is refracted from the second central area, and part of the light into the optical portion from the first annular area adjacent to the first central area is also refracted from the second central area. If the first annular area adjacent to the first central area is not completely projected onto the second central area, part of the light into the optical portion from the first annular area adjacent to the first central area is refracted from the second annular area.

In this way, the first central area and the second central area are combined to form one focal power, the first annular area adjacent to the first central area and the second central area are combined to form one focal power, the first annular area adjacent to the first central area and the second annular area are combined to form one focal power, another first annular area and the second annular area are combined to form one focal power, in which the another first annular area refers to the first annular area which is different from the first annular area adjacent to the first central area. The focal powers formed by the combination of the areas on the first surface and the areas of the second surface of the optical portion can be different from each other, so that the intraocular lens in this embodiment can provide at least four focal powers, that is, can have four focuses. Alternatively, the focal powers formed by the combination of the areas on the first surface and the areas of the second surface of the optical portion at least includes three different optical portions, for example, two of the four focal powers by the above combination are the same, so that intraocular lens at least includes three different focal powers, and at least includes three focuses.

Optionally, as a specific embodiment, the second central area is not completely projected onto the first central area, the first surface of the optical portion includes a proximal first annular area and a far first annular area, and the proximal first annular area is not completely projected onto the second central area. The proximal first annular area refers to the first annular area adjacent to the first central area, and the far first annular area refers to the first annular area adjacent to the proximal first annular area.

Specifically, the light from a first-distance object enters into the optical portion from the first central area, is refracted from the second central area, and converged to a first position on the optical axis of the optical portion; the light from a second-distance object enters into the optical portion from the proximal first annular area, is refracted from the second central area, and converged to a second position on the optical axis of the optical portion; the light from a third-distance object enters into the optical portion from the proximal first annular area, is refracted from the second annular area, and converged to a third position on the optical axis of the optical portion; the light from the second-distance object enters into the optical portion from the far first annular area, is refracted from the second annular area, and converged to the second position on the optical axis of the optical portion; a distance from the first-distance object to the center of the intraocular lens, a distance from the second-distance object to the center of the intraocular lens and a distance from the third-distance object to the center of the intraocular lens are different.

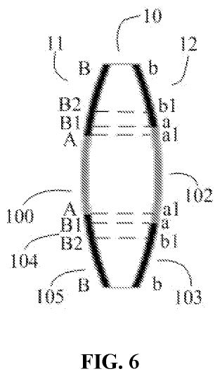

For example, referring to FIG. 5 and FIG. 6, FIG. 5 is a front view of the intraocular lens provided according to another embodiment of the present disclosure; and FIG. 6 is a side view of the intraocular lens shown in FIG. 5. As shown in the figures, the first surface 11 of the intraocular lens includes the first central area 100, a proximal first annular area 104 located on an inner side and a far first annular area 105 on an outer side, and the second surface 12 includes the second central area 102 and the second annular area 103. A radial size of the first central area 100 is less than a radial size of the second central area 102, so that the second central area 102 is not completely projected onto the first central area 100. A distance from an outer side edge of the proximal first annular area 104 to the center of the intraocular lens is greater than the radial size of the second central area 102, so that the proximal first annular area 104 is not completely projected onto the second central area 102.

Referring to FIG. 6, the first central area 100 is A-A area, the proximal first annular area 104 is A-B2 area, the far first annular area 105 is B2-B, the second central area 102 is a-a area, and the second annular area 103 is a-b area. It should be noted that the dotted lines in FIG. 6 are only used to indicate the corresponding relation of the areas of the first surface and the second surface of the optical portion 10, which is not to define the structure of the optical portion 10.

The light from a first-distance object enters into the optical portion 10 from the first central area 100, that is, from the A-A area, and is refracted from the second central area 102, specifically refracted from the al-al of the second central area 102, and converged to a first position on the optical axis of the optical portion 10.

The light from a second-distance object enters into the optical portion 10 from the proximal first annular area 104, specifically from the A-B1 area of the proximal first annular area 104, and is refracted from the al-a area of the second central area 102, and converged to a second position on the optical axis of the optical portion 10. That is, the second-distance object is imaged at the second position through the intraocular lens.

The light from a third-distance object enters into the optical portion 10 from the proximal first annular area 104, specifically from the B1-B2 of the proximal first annular area 104, and is refracted from the a-b1 area of the second annular area 103, and converged to a third position on the optical axis of the optical portion 10. That is, the third-distance object is imaged at the third position through the intraocular lens.

In addition, the light from the second-distance object enters into the optical portion 10 from the far first annular area 105, that is, from the B2-B area, and is refracted from the b1-b area of the second annular area 103, and converged to the second position on the optical axis of the optical portion 10. Therefore, the intraocular lens in this embodiment has three focuses, which can image the first-distance object, the second-distance object and the third-distance object, and can provide distant vision, medium vision and proximal vision.

As another optional embodiment, the first central area is not completely projected onto the second central area, the light from a first-distance object enters into the optical portion from the first central area, and is refracted from the second central area, and converged to a first position on the optical axis of the optical portion; the light from a second-distance object enters into the optical portion from the first central area, and is refracted from the second annular area, and converged to a second position on the optical axis of the optical portion. A distance from the first-distance object to the center of the intraocular lens is different from a distance from the second-distance object to the center of the intraocular lens. The intraocular lens in this embodiment has two focuses, which can image the first-distance object and the second-distance object.

In this embodiment, the magnitude relation between the distance from the first-distance object to the center of the intraocular lens and the distance from the second-distance object to the center of the intraocular lens is not limited. For example, a minimum distance of the first distance range may be greater than a maximum distance of the second distance range, that is, the distance from the first-distance object to the center of the intraocular lens is greater than the distance from the second-distance object to the center of the intraocular lens.

Furthermore, the light from a third-distance object enters into the optical portion from the first annular area, and is refracted from the second annular area, converged to a third position on the optical axis of the optical portion. A distance from the third-distance object to the center of the intraocular lens is different from the distance from the second-distance object to the center of the intraocular lens as well as the distance from the first-distance object to the center of the intraocular lens. The intraocular lens in this embodiment has three focuses, which can image the first-distance object, the second-distance object and the third-distance object, and can provide distant vision, medium vision and proximal vision. In this embodiment, the magnitude relation among the distance from the third-distance object to the center of the intraocular lens, the distance from the first-distance object to the center of the intraocular lens and the distance from the second-distance object to the center of the intraocular lens is not limited. For example, the minimum distance of the first distance range may be greater than the maximum distance of the second distance range, a minimum distance of the second distance range may be greater than a maximum distance of the third distance range, that is, the distance from the first-distance object to the center of the intraocular lens is greater than the distance from the second-distance object to the center of the intraocular lens, and the distance from the second-distance object to the center of the intraocular lens is greater than the distance from the third-distance object to the center of the intraocular lens.

Optionally, as another optional embodiment, the first central area is not completely projected onto the second central area, the second surface of the optical portion includes at least two second annular areas, and the second annular area adjacent to the second central area is not completely projected onto the first central area, and the at least two second annular areas have different focal powers. The first central area is not completely projected onto the second central area, that is, at least part of the first central area is projected onto the second annular area adjacent to the second central area, so that part of the light into the optical portion from the first central area is refracted from the second central area, and another part of the light is refracted from the second annular area adjacent to the second central area. If the second annular area adjacent to the second central area is not completely projected onto the first central area, part of the light into the optical portion from the first annular area is refracted from the second annular area adjacent to the second central area.

In that case, the first central area and the second central area are combined to form one focal power, the first central area and the second annular area adjacent to the second central area are combined to form one focal power, the first annular area and the second annular area adjacent to the second central area are combined to form one focal power, the first annular area and the other second annular area are combined to form one focal power, in which the another second annular area refers to the second annular area which is different from the second annular area adjacent to the second central area. The focal powers formed by the combination of the areas on the first surface and the areas on the second surface of the optical portion can be different from each other, so that the intraocular lens in this embodiment can provide at least four focal powers, that is, can have four focuses. Alternatively, the focal powers formed by the combination of the areas on the first surface and the areas on the second surface of the optical portion at least includes three different optical portions, for example, two focal powers of the four focal powers formed by the above combination are the same, so that the intraocular lens at least includes three different focal powers, and at least includes three focuses.

Optionally, as another optional embodiment, the first central area is not completely projected onto the second central area, the second surface of the optical portion includes a proximal second annular area and a far second annular area, and the proximal second annular area is not completely projected onto the first central area. The proximal second annular area refers to the second annular area adjacent to the second central area, and the far second annular area refers to the second annular area adjacent to the proximal second annular area.

Specifically, the light from a first-distance object enters into the optical portion from the first central area, and is refracted from the second central area, and converged to a first position on the optical axis of the optical portion; the light from a second-distance object enters into the optical portion from the first central area, and is refracted from the proximal second annular area, and converged to a second position on the optical axis of the optical portion; the light from a third-distance object enters into the optical portion from the first annular area, and is refracted from the proximal second annular area, and converged to a third position on the optical axis of the optical portion; the light from the second-distance object enters into the optical portion from the first annular area, and is refracted from the far second annular area, and converged to the second position on the optical axis of the optical portion. A distance from the first-distance object to the center of the intraocular lens, a distance from the second-distance object to the center of the intraocular lens and a distance from the third-distance object to the center of the intraocular lens are all different. The intraocular lens in this embodiment has three focuses, which can image the first-distance object, the second-distance object and the third-distance object, and can provide distant vision, medium vision and proximal vision.

Preferably, in the above embodiments, it can be that the focal power of the first annular area gradually changes, or it can be that the focal power of the first annular area from an outer side to an inner side gradually increases. For example, the focal power of an outermost side edge of the first annular area is close to the focal power of the first central area, and the focal power gradually increases from the outer side to the inner side, and the focal power increases to the designed focal power of the first annular area when approaching an inner side edge of the first annular area.

Preferably, in the above embodiment, it can be that the focal power of the second annular area gradually changes. It can be that the focal power of the second annular area from an outer side to an inner side gradually increases. For example, the focal power of the second annular area gradually increases, the focal power of an outermost edge of the second annular area is close to the focal power of the second central area, and the focal power gradually increases the outer side to the inner side, and the focal power increases to the designed focal power of the second annular area when approaching an inner side edge of the second annular area. For example, for the intraocular lens shown in FIGS. 1 to 4, the first-distance object is a long-distance object, the second-distance object is a medium-distance object, and the third-distance object is a short-distance object, and if the focal power of the second annular area gradually increases from the outer side to the inner side as described above, the second annular area has the medium-distance object on the outer side, the short-distance object on the inner side, and thus this design dilates the pupil to greater than 3.6 mm in dark environments, such as indoor environments, which increases the light energy of the medium-distance object into the pupil, and the light energy of the short-distance object does not need to be too much.

Preferably, in the above embodiments, an outer side edge of the first central area has a same height as an inner side edge of the first annular area, so that the first central area and the first annular area of the first surface form a zero-step height transition. If the first surface includes multiple first annular areas, heights of connecting edges of the two adjacent first annular areas are the same, and thus the zero-step height transition is formed on the first surface of the intraocular lens.

Preferably, in the above embodiments, an outer side edge of the second central area has a same height as an inner side edge of the second annular area, so that the second central area and the second annular area of the second surface form a zero-step height transition. If the second surface includes multiple second annular areas, heights of connecting edges of the two adjacent second annular areas are the same, so that the zero-step height transition is formed on the second surface of the intraocular lens.

The conventional refractive multifocal intraocular lens has uneven refractive rings on its surface, and the diffractive multifocal intraocular lens has uneven diffractive rings on its surface, and the uneven structures on the surface of the intraocular lens are easy to cause glare and halo. However, in the intraocular lens in this embodiment, the zero-step height transition is formed on the first surface or the second surface, which can reduce the phenomenon of glare and halo, enable the smooth surface and natural transition of the surface of the intraocular lens, reduce light scattering, and improve the contrast sensitivity and the light energy utilization.

In the above embodiments, the first central area of the first surface may be spherical or aspherical, and the first annular area of the first surface may be spherical or aspherical. In the above embodiments, the second central area of the second surface may be spherical or aspherical, and the second annular area of the second surface may be spherical or aspherical. The surface of the optical portion is aspherical, and the focal power of the surface can be gradually changed by using the aspherical surface.

According to International Standard ISO 11979-2 “Ophthalmic Implants-Intraocular lenses-Part 2: Optical Properties and Test Methods”, the calculation formula of the focal power of the intraocular lens is given as follows:

D=D_{f}+D_{b}−(t_{c}/n_{IOL})·D_{f}·D_{b}; (1)

where, D is the focal power of the intraocular lens, in diopter (D), D_{f} is the focal power of an anterior surface of the intraocular lens; D_{b} is the focal power of a posterior surface of the intraocular lens, t_{c} represents a central thickness of the intraocular lens, in meters (m), n_{IOL} represents the refractive index of the optical material of the intraocular lens.

The formula for calculating the focal power of the anterior surface of the intraocular lens is:

D_{f}=(n_{IOL}−n_{med})/r_{f}; (2)

where, r_{f} represents the curvature radius of the anterior surface of the intraocular lens, in meters (m), and n_{med} represents the refractive index of the media surrounding the intraocular lens. For example, the media surrounding the intraocular lens is aqueous humor and vitreous, and the refractive index of both aqueous humor and vitreous is 1.336.

The formula for calculating the focal power of the posterior surface of the intraocular lens is:

D_{b}=(n_{med}−n_{IOL})/r_{b}; (3)

where, r_{b} represents the curvature radius of the anterior surface of the intraocular lens, in meters (m), and n_{med} represents the refractive index of the media surrounding the intraocular lens.

In a specific embodiment, the intraocular lens shown in FIGS. 1 to 4 is made of acrylate material with a refractive index of 1.5385, the central thickness of the intraocular lens is 0.001m, a diameter of the first central area 100 of the first surface of 1.96 mm, a focal power of +11.13D, and a curvature radius r_{A} of 18.194 mm. The first annular area 101 refers to the area with a diameter ranging from 1.96 mm to 6 mm, a focal power of +12.86D, and a curvature radius r_{B} of 15.747 mm. The second central area 102 of the second surface 12 has a diameter of 2.56 mm, a focal power of +10.95D, and a curvature radius rₐ of 18.493 mm. The second annular area 103 refers to the area with a diameter ranging from 2.56 mm to 6 mm, an focal power of +12.44D, and a curvature radius r_{b} of 16.278 mm. The focal power on each surface and the combined focal powers of the intraocular lens are shown in Table 1 below

TABLE 1

| | | | | Combined | | |
|---|---|---|---|---|---|---|
| | Focal | | Focal | focal | | |
| First | power | Second | power | power | Diameter | |
| surface | (D) | surface | (D) | (D) | (mm) | |
| B1-B | 12.86 | a-b | 12.44 | 25.20 | 2.56 mm < | Proximal |
| | | | | | d ≤ 6 mm | focus |
| A-B1 | 12.86 | a1-a | 10.95 | 23.72 | 1.96 mm < | Medium |
| | | | | | d ≤ 2.56 mm | focus |
| A-A | 11.13 | a1-a1 | 10.95 | 22.00 | d ≤ 1.96 mm | Far focus |
| A-B1 | 12.86 | a1-a | 10.95 | 23.72 | 1.96 mm < | Medium |
| | | | | | d ≤ 2.56 mm | focus |
| B1-B | 12.86 | a-b | 12.44 | 25.20 | 2.56 mm < | Proximal |
| | | | | | d ≤ 6 mm | focus |

The light energy distribution of the intraocular lens is as follows: if the standard pupil diameter is 3 mm, a circle area with a diameter of 3 mm is 7.065 mm², a circle area with a diameter of 2.56 mm is 5.145 mm², and a circle area with a diameter of 1.96 mm is 3.016 mm². The area of the optical portion of the intraocular lens for viewing long-distance objects is 3.016 mm², the optical energy of distant vision accounts for 42.68%; the area of the optical portion of the intraocular lens for viewing medium-distance objects is 5.145 mm²-3.016 mm²=2.219 mm², and the optical energy of medium vision accounts for 30.13%; the area of the optical portion of the intraocular lens for viewing short-distance objects is 7.065 mm²-5.145 mm²=1.920 mm², and the optical energy of proximal vision accounts for 27.18%. The light energy distribution of the combined refractive trifocal intraocular lens can vary with the dilatation or shrinkage of the pupil. In the room, when the light is dark and the pupils become greater, the area of the optical portion of the intraocular lens for viewing short-distance objects can apparently become greater, and the proximal vision for reading can be improved significantly.

In another specific embodiment, the intraocular lens as shown in FIG. 5 and FIG. 6 is made of acrylate material with a refractive index of 1.5385. A diameter of the first central area 100 of the first surface 11 is 1.96 mm, a focal power is +11.13D, and a curvature radius r_{A} is 18.194 mm. The proximal first annular area 104 refers to the area with a diameter ranging from 1.96 mm to 3.6 mm, an focal power of +12.86D, and a curvature radius r_{B} of 15.747 mm. The far first annular area 105 refers to the area with a diameter ranging from 3.6 mm to 6 mm, an focal power of +11.13D, and a curvature radius r_{B} of 18.194 mm.

The second central area 102 of the second surface 12 has a diameter of 2.56 mm, a focal power of +10.95D, and a curvature radius rₐ of 18.493 mm. The second annular area 103 refers to the area with a diameter ranging from 2.56 mm to 6 mm, an focal power of +12.44D, and a curvature radius r_{b} of 16.278 mm. The focal power on each surface and the combined focal powers of the intraocular lens are shown in Table 2 below

TABLE 2

| | | | | Com- | | |
|---|---|---|---|---|---|---|
| | | | | bined | | |
| | Focal | | Focal | focal | | |
| First | power | Second | power | power | | |
| surface | (D) | surface | (D) | (D) | Diameter (mm) | |
| B2-B | 11.13 | b1-b | 12.44 | 23.72 | 3.6 mm < | Medium |
| | | | | | d ≤ 6 mm | focus |
| B1-B2 | 12.86 | a-b1 | 12.44 | 25.20 | 2.56 mm < | Proximal |
| | | | | | d ≤ 3.6 mm | focus |
| A-B1 | 12.86 | a1-a | 10.95 | 23.72 | 1.96 mm < | Medium |
| | | | | | d ≤ 2.56 mm | focus |
| A-A | 11.13 | a1-a1 | 10.95 | 22.00 | d ≤ 1.96 mm | Far focus |
| A-B1 | 12.86 | a1-a | 10.95 | 23.72 | 1.96 mm < | Medium |
| | | | | | d ≤ 2.56 mm | focus |
| B1-B2 | 12.86 | a-b1 | 12.44 | 25.20 | 2.56 mm < | Proximal |
| | | | | | d ≤ 3.6 mm | focus |
| B2-B | 11.13 | b1-b | 12.44 | 23.72 | 3.6 mm < | Medium |
| | | | | | d≤ 6 mm | focus |

Optionally, the intraocular lens in this embodiment may be made of acrylate polymer (acrylate for short). The acrylate has good light transmittance with refractive index as high as 1.5385 at 37° C., so the intraocular lens is thinner and lighter than the intraocular lens made of other material.

Preferably, the intraocular lens in this embodiment can be processed with an ultra-high precision lathe, and the processing accuracy of the ultra-high precision lathe should reach 1 m. Acrylate round embryo is used as the raw material for turning, and a diamond knife is selected. The anterior surface of the intraocular lens is machined according to the curvature radius from r_{A} to r_{B}, and the posterior surface of the intraocular lens is machined according to the curvature radius from rₐ to r_{b}. Finally, a high precision milling machine is used to process the contour and loop of intraocular lens.

The intraocular lens provided according to the present disclosure has been described in detail hereinbefore. The principle and embodiments of the present disclosure are described through specific examples herein. The description of the above-described embodiments is merely used to facilitate understanding the method and core idea of the present disclosure. It should be pointed out that, various improvements and modifications can be made by those skilled in the art without departing from the principle of the present disclosure, and these improvements and modifications should fall within the protection scope of the present disclosure.

1. An intraocular lens, comprising an optical portion, wherein a first surface of the optical portion comprises a first central area and at least one first annular area, and the first central area and the at least one first annular area have different focal powers;
a second surface of the optical portion comprises a second central area and at least one second annular area, the second central area and the at least one second annular area have different focal powers, and the first central area and the second central area are not completely projected onto each other, so that light enters into the optical portion from the first surface, and is refracted from the second surface, and converged to at least three different positions on an optical axis of the optical portion.

2. The intraocular lens according to claim 1, wherein the light from a first-distance object enters into the optical portion from the first central area, and is refracted from the second central area, and converged to a first position on the optical axis of the optical portion;
if the second central area is not completely projected onto the first central area, the light from a second-distance object enters into the optical portion from the at least one first annular area, and is refracted from the second central area, and converged to a second position on the optical axis of the optical portion;
if the first central area is not completely projected onto the second central area, the light from the second-distance object enters into the optical portion from the first central area, and is refracted from the at least one second annular area, and converged to the second position on the optical axis of the optical portion;
a distance from the first-distance object to the center of the intraocular lens is different from a distance from the second-distance object to the center of the intraocular lens.

3. The intraocular lens according to claim 2, wherein the distance from the first-distance object to the center of the intraocular lens is greater than the distance from the second-distance object to the center of the intraocular lens.

4. The intraocular lens according to claim 2, wherein the light from a third-distance object enters into the optical portion from the at least one first annular area, and is refracted from the at least one second annular area, converged to a third position on the optical axis of the optical portion, and a distance from the third-distance object to the center of the intraocular lens is different from both the distance from the second-distance object to the center of the intraocular lens and the distance from the first-distance object to the center of the intraocular lens.

5. The intraocular lens according to claim 4, wherein the distance from the first-distance object to the center of the intraocular lens is greater than the distance from the second-distance object to the center of the intraocular lens, and the distance from the second-distance object to the center of the intraocular lens is greater than the distance from the third-distance object to the center of the intraocular lens.

6. The intraocular lens according to claim 4, wherein a distance from the second position to the center of the intraocular lens is less than a distance from the first position to the center of the intraocular lens, and a distance from the third position to the center of the intraocular lens is less than a distance from the second position to the center of the intraocular lens.

7. The intraocular lens according to claim 1, wherein the second central area is not completely projected onto the first central area, the first surface of the optical portion comprises at least two first annular areas, the first annular area adjacent to the first central area is not completely projected onto the second central area, and each of the at least two first annular areas has different focal powers;
or, the first central area is not completely projected onto the second central area, the second surface of the optical portion comprises at least two second annular areas, the second annular area adjacent to the second central area is not completely projected onto the first central area, and each of the at least two second annular areas has different focal powers.

8. The intraocular lens according to claim 7, wherein the second central area is not completely projected onto the first central area, the first surface of the optical portion comprises a proximal first annular area and a far first annular area, and the proximal first annular area is not completely projected onto the second central area;
the light from a first-distance object enters into the optical portion from the first central area, and is refracted from the second central area, and converged to a first position on the optical axis of the optical portion;
the light from a second-distance object enters into the optical portion from the proximal first annular area, and is refracted from the second central area, and converged to a second position on the optical axis of the optical portion;
the light from a third-distance object enters into the optical portion from the proximal first annular area, and is refracted from the second annular area, and converged to a third position on the optical axis of the optical portion;
the light from the second-distance object enters into the optical portion from the far first annular area, and is refracted from the second annular area, and converged to the second position on the optical axis of the optical portion;
a distance from the first-distance object to the center of the intraocular lens, a distance from the second-distance object to the center of the intraocular lens and a distance from the third-distance object to the center of the intraocular lens are all different.

9. The intraocular lens according to claim 7, wherein the first central area is not completely projected onto the second central area, the second surface of the optical portion comprises a proximal second annular area and a far second annular area, and the proximal second annular area is not completely projected onto the first central area;
the light from a first-distance object enters into the optical portion from the first central area, and is refracted from the second central area, and converged to a first position on the optical axis of the optical portion;
the light from a second-distance object enters into the optical portion from the first central area, and is refracted from the proximal second annular area, and converged to a second position on the optical axis of the optical portion;
the light from a third-distance object enters into the optical portion from the first annular area, and is refracted from the proximal second annular area, and converged to a third position on the optical axis of the optical portion;
the light from the second-distance object enters into the optical portion from the first annular area, and is refracted from the far second annular area, and converged to the second position on the optical axis of the optical portion;
a distance from the first-distance object to the center of the intraocular lens, a distance from the second-distance object to the center of the intraocular lens and a distance from the third-distance object to the center of the intraocular lens are all different.

10. The intraocular lens according to any one of claim 1, wherein a radial size of the first central area is less than a radial size of the second central area, so that the second central area is not completely projected onto the first central area;
or, a radial size of the first central area is greater than a radial size of the second central area, so that the first central area is not completely projected onto the second central area.

11. The intraocular lens according to any one of claim 2, wherein a radial size of the first central area is less than a radial size of the second central area, so that the second central area is not completely projected onto the first central area;
or, a radial size of the first central area is greater than a radial size of the second central area, so that the first central area is not completely projected onto the second central area.

12. The intraocular lens according to any one of claim 3, wherein a radial size of the first central area is less than a radial size of the second central area, so that the second central area is not completely projected onto the first central area;
or, a radial size of the first central area is greater than a radial size of the second central area, so that the first central area is not completely projected onto the second central area.

13. The intraocular lens according to any one of claim 4, wherein a radial size of the first central area is less than a radial size of the second central area, so that the second central area is not completely projected onto the first central area;
or, a radial size of the first central area is greater than a radial size of the second central area, so that the first central area is not completely projected onto the second central area.

14. The intraocular lens according to any one of claim 5, wherein a radial size of the first central area is less than a radial size of the second central area, so that the second central area is not completely projected onto the first central area;
or, a radial size of the first central area is greater than a radial size of the second central area, so that the first central area is not completely projected onto the second central area.

15. The intraocular lens according to any one of claim 6, wherein a radial size of the first central area is less than a radial size of the second central area, so that the second central area is not completely projected onto the first central area;
or, a radial size of the first central area is greater than a radial size of the second central area, so that the first central area is not completely projected onto the second central area.

16. The intraocular lens according to any one of claim 7, wherein a radial size of the first central area is less than a radial size of the second central area, so that the second central area is not completely projected onto the first central area;
or, a radial size of the first central area is greater than a radial size of the second central area, so that the first central area is not completely projected onto the second central area.

17. The intraocular lens according to any one of claim 8, wherein a radial size of the first central area is less than a radial size of the second central area, so that the second central area is not completely projected onto the first central area;
or, a radial size of the first central area is greater than a radial size of the second central area, so that the first central area is not completely projected onto the second central area.

18. The intraocular lens according to claim 1, wherein a focal power of the at least one first annular area from an outer side to an inner side gradually increases, or, a focal power of the at least one second annular area from an outer side to an inner side gradually increases.

19. The intraocular lens according to claim 1, wherein an outer side edge of the first central area has a same height as an inner side edge of the least one first annular area, or, an outer side edge of the second central area has a same height as an inner side edge of the at least one second annular area.

20. The intraocular lens according to claim 1, wherein the first central area is spherical or aspherical, the at least one first annular area is spherical or aspherical, the second central area is spherical or aspherical, and the at least one second annular area is spherical or aspherical.
